(19) United States
(12) Patent Application Publication
(10) Pub. No.: US 2009/0105575 A1
(43) Pub. Date: Apr. 23, 2009

(54) BIO-SENSING SYSTEM, MICRO-SENSING ELEMENT AND MANUFACTURING METHOD THEREOF

(76) Inventors: Chu-Lin FAN, Tainan City (TW); Lung-Jieh Yang, Taipei County (TW); Pen-Li Lu, Taipei City (TW); Chij-Wann Lin, Taipei City (TW); Fu-Shan Jaw, Taipei (TW)

Correspondence Address: BIRCH STEWART KOLASCH & BIRCH, PO BOX 747, FALLS CHURCH, VA, 22040-0747, US

(21) Appl. No.: 12/237,462

(22) Filed: Sep. 25, 2008

(30) Foreign Application Priority Data
Oct. 19, 2007 (TW) 096139227

(51) Int. Cl. A61B 5/04; B05D 5/12

(52) U.S. Cl. 600/372; 427/2.12

(57) ABSTRACT

A micro-sensing element includes a substrate, a micro-sensing structure and a covering material. The micro-sensing structure has a plurality of conductive channels disposed on the substrate. Each conductive channel includes a sensing part, a conductive wire and an electrode. The sensing part is electrically connected with the electrode through the conductive wire. The covering material covers the substrate and the conductive wires, and each of the sensing parts and each of the electrodes are exposed out of the covering material. A bio-sensing system and a manufacturing method of the micro-sensing element are also disclosed.

### CROSS REFERENCE TO RELATED APPLICATIONS

This Non-provisional application claims priority under 35 U.S.C. §119(a) on Patent Application No(s). 096139227 filed in Taiwan, Republic of China on Oct. 19, 2007, the entire contents of which are hereby incorporated by reference.

### BACKGROUND OF THE INVENTION

1. Field of Invention

The invention relates to a micro-sensing element and a manufacturing method thereof. More particularly, the invention relates to a micro-sensing element and a manufacturing method thereof that utilize the micro-electro-mechanical-system (MENS) manufacturing technology.

2. Related Art

A micro-sensing element is for sensing a nervous signal of an animal, and thus becomes an indispensable tool in the nervous physiological research. In order to embed the micro-sensing element into a live biometric body to observe the nervous signals of the wakened animal corresponding to the learning and the behavior thereof, the good biometric compatibility has to be provided. Typically, the so-called micro-sensing element does not have the biometric compatibility, and an additional biometric compatibility material has to be provided for covering. The covering material has to prevent the improper aftereffect from being induced and to avoid the erosion of the salinity of the biometric body fluid, and has to facilitate the long-term embedding in conjunction with different biometric physiological cycles.

The micro-sensing element includes a silicon substrate with conductive wires and is covered by parylene according to the MENS manufacturing technology. The parylene has the protection effects on the medical treatment product in the aspects of the germproof property and the oxidation resistance, and is proved as having the good biometric compatibility. In the prior art, only one side of the micro-sensing element can be covered by the parylene. However, the one-side covering process still cannot completely cover the micro-sensing element having the substrate made of silicon, and only one side of the silicon substrate is covered with other surfaces being exposed out of the micro-sensing element. Thus, the silicon substrate may contact with the biometric body and is not suitable for the application of embedding into the biometric body for a long time. Accordingly, the biometric compatibility still has to be improved.

In order to make the micro-sensing element having the silicon substrate be embedded into the biometric body for a long time, the research on the technology of completely covering the micro-sensing element has become an important issue in the MENS manufacturing technology.

Therefore, it is an important subject to provide a micro-sensing element, a manufacturing method thereof and a bio-sensing system using the micro-sensing element in order to solve the above-mentioned problems and thus to enhance the biometric compatibility.

### SUMMARY OF THE INVENTION

In view of the foregoing, the invention is to provide the full covering technology to enhance the biometric compatibility of a micro-sensing element and a manufacturing method of the micro-sensing element, and a bio-sensing system applying the micro-sensing element.

To achieve the above, the invention discloses a micro-sensing element including a substrate, a micro-sensing structure and a covering material. The micro-sensing structure has a plurality of conductive channels disposed on the substrate. Each conductive channel includes a sensing part, a conductive wire and an electrode. The sensing part is electrically connected with the electrode through the conductive wire. The covering material covers the substrate and the conductive wire, and each sensing part and each electrode are exposed out of the covering material.

To achieve the above, the invention also discloses a manufacturing method of a micro-sensing element including the steps of: providing a substrate; forming at least one micro-sensing structure on the substrate, wherein the micro-sensing structure has a plurality of conductive channels, each of the conductive channels comprises a sensing part, a conductive wire and an electrode, and the sensing part is electrically connected with the electrode through the conductive wire; covering the substrate and the conductive channels by a covering material; and partially removing the covering material to make each of the sensing parts and each of the electrodes be exposed out of the covering material.

In addition, the invention further discloses a bio-sensing system including a micro-sensing element and at least one electronic assembly. The micro-sensing element has a substrate, a micro-sensing structure and a covering material. The micro-sensing structure has a plurality of conductive channels disposed on the substrate. Each conductive channel includes a sensing part, a conductive wire and an electrode. The sensing part is electrically connected with the electrode through the conductive wire. The covering material covers the substrate and the conductive wires, and each sensing part and each electrode are exposed out of the covering material. The electronic assembly is electrically connected with the electrodes.

The conductive channels are integrally formed in a silicon substrate. That is, each sensing part, each electrode and each conductive wire are integrally formed. An insulating layer is disposed between the conductive channels and the substrate. The material of the conductive channel is metal or an alloy, wherein the metal or alloy includes gold (Au), nickel (Ni), chromium (Cr), iridium (Ir), palladium (Pd), niobium (Nb), titanium (Ti), platinum (Pt) or any other substance. The sensing parts and electrodes are exposed out of the covering material through an opening or by protruding from the opening.

The covering material is a polymeric material having the biometric compatibility and may be selected from the group consisting of parylene, gelatin and combinations thereof. The polymeric material may be removed by way of tearing, etching or laser removing. It is possible to use the laser to cut the micro-sensing element according to the actual requirement to have a changeable specification according to the changeable biometric sensing condition.

As mentioned above, in the invention, the covering material with the biometric compatibility completely covers the micro-sensing element, and only the sensing parts are exposed out of the covering material to provide the function of biometric sensing. In addition, the electrodes are exposed out of the covering material to provide the function of transferring the sensed signal. Compared with the prior art, the micro-sensing element of the invention has the good biometric compatibility, and can be embedded into the biometric body to sense the biometric body for a long term.

### BRIEF DESCRIPTION OF THE DRAWINGS

The invention will become more fully understood from the detailed description and accompanying drawings, which are given for illustration only, and thus are not limitative of the present invention, and wherein:

FIG. 1 is a flow chart showing a manufacturing method of a micro-sensing element according to a preferred embodiment of the invention;

FIGS. 2A to 2D are schematic illustrations showing structures of the micro-sensing element corresponding to steps of the manufacturing method of FIG. 1;

FIG. 3 is a schematic illustration showing a micro-sensing element according to the preferred embodiment of the invention; and

FIG. 4 is a schematic illustration showing a bio-sensing system according to the preferred embodiment of the invention.

### DETAILED DESCRIPTION OF THE INVENTION

The present invention will be apparent from the following detailed description, which proceeds with reference to the accompanying drawings, wherein the same references relate to the same elements.

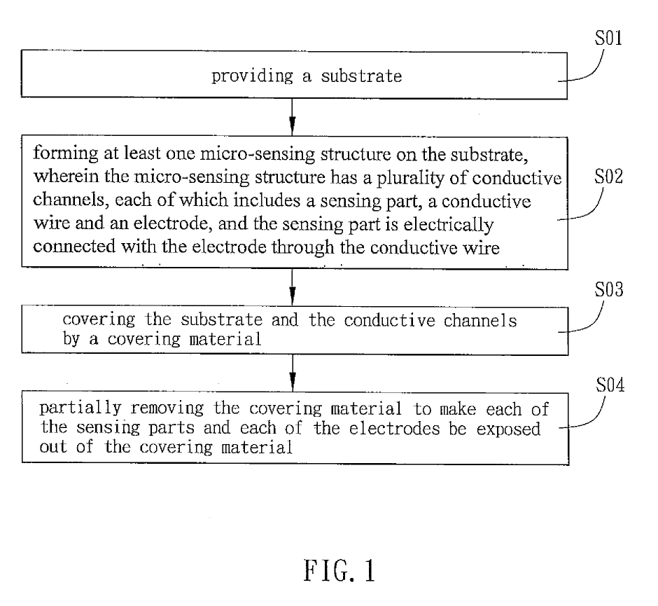

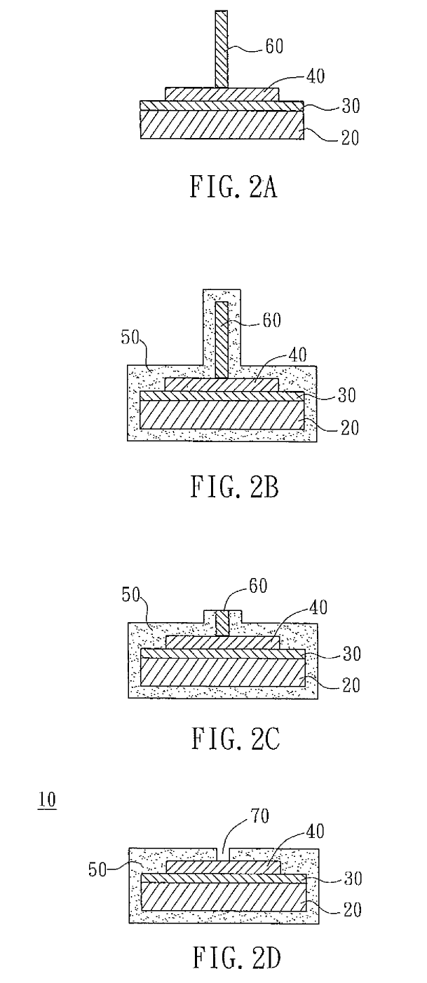

Referring to FIG. 1, a manufacturing method of a micro-sensing element 10 according to a preferred embodiment of the invention includes steps S01 to S04. Referring to FIGS. 2A to 2D, the manufacturing method of the micro-sensing element 10 will be described in detail.

As shown in FIGS. 1 and 2A, a substrate 20 is provided in the step S01. An insulating layer 30 may be formed on the substrate 20 in advance to prevent ions in the substrate 20 from migrating to an element thereon and thus to damage the element. The material of the insulating layer 30 is silicon oxide (SiO), silicon nitride (SiN) or silicon oxy-nitride (SiON), for example.

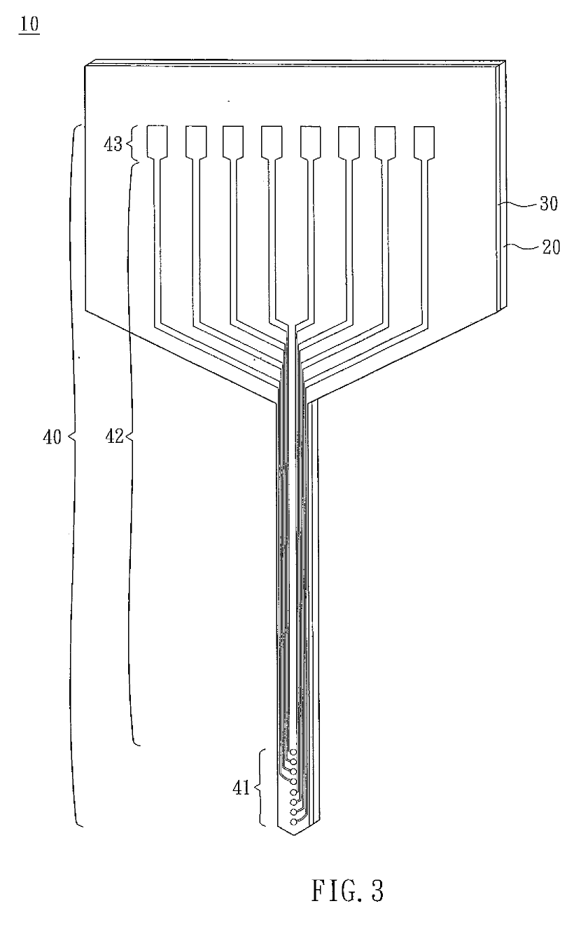

In the step 802, at least one micro-sensing structure 40 is formed on the substrate 20. The micro-sensing structure 40 has a plurality of conductive channels each including a sensing part 41, a conductive wire 42 and an electrode 43, as shown in FIG. 3. The sensing part 41 is electrically connected with the electrode 43 through the conductive wire 42.

In this embodiment, a conductive part 60 may further be formed adjacent to each of the sensing parts 41, and the material of the conductive part 60 may be metal or an alloy, such as aluminum or an aluminum alloy. The conductive part 60 may be combined with an end portion of the conductive wire 42 by way of wire bonding, for example.

The step of forming the conductive wires 42 includes the sub-steps of: forming a resist layer (not shown) on the insulating layer 30; exposing and developing the resist layer to form a patterned resist layer; forming a conductive layer (not shown) on the patterned resist layer, wherein the conductive layer partially covers the resist layer and partially covers the insulating layer 30; and removing the patterned resist layer to form the conductive wires 42.

Alternatively, the step of forming the conductive wires 42 includes the sub-steps of: forming a conductive layer (not shown) on the insulating layer 30; forming a resist layer (not shown) on the conductive layer; exposing and developing the resist layer to form the patterned resist layer; etching the conductive layer, which is not covered by the patterned resist layer, to leave the conductive wires 42; and removing the patterned resist layer to form the conductive wires 42.

As shown in FIGS. 1 and 2B, a covering material 50 covers the substrate 20 and the conductive channels in the step S03. That is, the covering material 50 covers the substrate 20, the sensing parts 41, the conductive wires 42 and the electrodes 43. The covering material 50 may be a polymeric material selected from the group consisting of parylene, gelatin and combinations thereof.

As shown in FIGS. 1 and 2C, the covering material 50 is partially removed in the step S04 so that each of the sensing parts 41 and each of the electrodes 43 are exposed out of the covering material 50. The polymeric material may be removed by way of tearing, etching or laser removing according to the actual requirement. Partially removing the conductive part 60 can simultaneously partially remove the covering material 50. The conductive part 60 may be partially removed by way of breaking or snipping. The sensing part 41 projects and is exposed outside according to the residual conductive part 60. The sensing parts 41 are exposed out of the covering material 50 to provide the function of biometric sensing. The electrodes 43 are exposed out of the covering material 50 to provide the function of transferring the sensed signal. The manufacturing of the micro-sensing element 10 is finished according to the above-mentioned steps.

Alternatively, as shown in FIG. 2D, the residual conductive part 60 is removed to leave a space serving as an opening 70 to expose each of the sensing parts 41 to provide the function of biometric sensing. The residual conductive part 60 may be removed by way of etching, such as wet etching.

Referring to FIG. 3, the micro-sensing element 10 according to the preferred embodiment of the invention includes the substrate 20 and the micro-sensing structure 40. As shown in FIG. 2D, the micro-sensing element 10 is covered by the covering material 50, the insulating layer 30 may be disposed on the substrate 20, and then the micro-sensing structure 40 may be formed on the insulating layer 30. The exterior of the micro-sensing element 10 may be determined according to the actual requirement by way of laser cutting.

The micro-sensing structure 40 has the plurality of conductive channels disposed on the substrate 20. Each of the conductive channels includes the sensing part 41, the conductive wire 42 and the electrode 43. The sensing part 41 is electrically connected with the electrode 43 through the conductive wire 42. The covering material 50 covers the substrate 20 and the conductive wires 42, and each of the sensing parts 41 and each of the electrodes 43 are exposed out of the covering material 50. The sensing pads 41 are exposed out of the covering material 50 to provide the function of biometric sensing. The electrodes 43 are exposed out of the covering material 50 to provide the function of transferring the sensed signal.

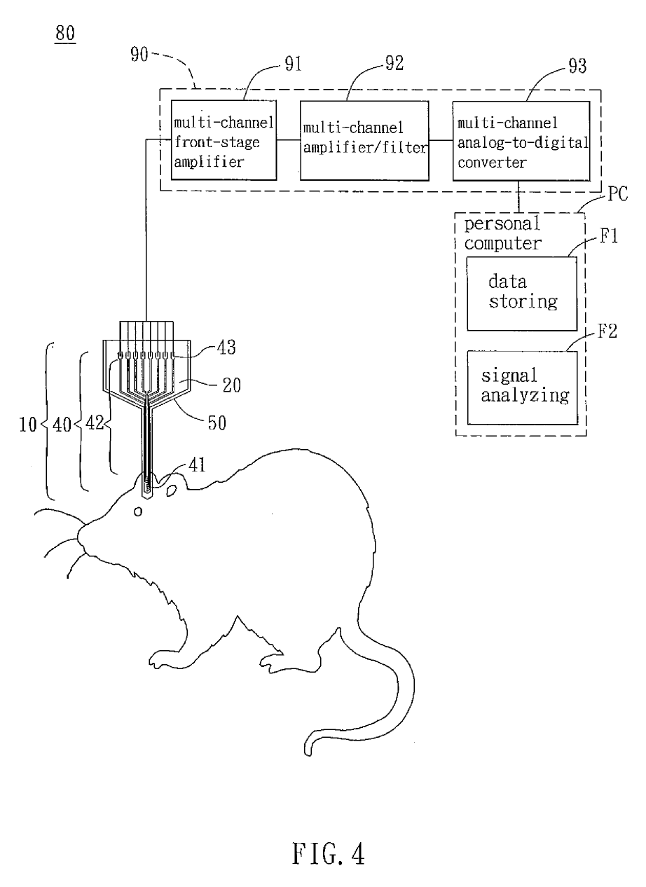

Referring to FIG. 4, a bio-sensing system 80 applied to the micro-sensing element 10 according to the preferred embodiment of the invention includes the micro-sensing element 10 and at least one electronic assembly 90. The micro-sensing element 10 has the substrate 20, the micro-sensing structure 40 and the covering material 50. The micro-sensing structure 40 has the plurality of conductive channels disposed on the substrate 20. Each of the conductive channels includes the sensing part 41, the conductive wire 42 and the electrode 43. The electronic assembly 90 is electrically connected with the electrodes 43. The electronic assembly 90 may be selected from the group consisting of an amplifier, a filter, an analog-to-digital converter, a processor, a storage and combinations thereof, and the electronic assembly 90 may be disposed on a circuit board.

In this embodiment, the electronic assembly 90 includes a multi-channel front-stage amplifier 91, a multi-channel amplifier/filter 92 and a multi-channel analog-to-digital converter 93. The sensing parts 41 receive a sensed signal, which is transferred to the electronic assembly 90 through the electrodes 43. That is, the sensed signal is first transferred to the multi-channel front-stage amplifier 91, which amplifies the sensed signal, and the amplified sensed signal is transferred to the multi-channel amplifier/filter 92, which amplifies and filters the amplified sensed signal to generate an analog signal. Then, the multi-channel analog-to-digital converter 93 converts the analog signal into a digital signal. Finally, the digital signal may be transferred to a personal computer PC for performing functions of data storing F1 and signal analyzing F2.

In summary, the micro-sensing element, the manufacturing method thereof and the bio-sensing system applying the micro-sensing element according to the invention have the following features. First, the covering material with the biometric compatibility completely covers the micro-sensing element, and only the sensing parts are exposed out of the covering material to provide the function of biometric sensing. Second, the electrodes are exposed out of the covering material to provide the function of transferring the sensed signal. Compared with the prior art, the micro-sensing element of the invention has the good biometric compatibility, and can be embedded into the biometric body to sense the biometric body for a long term.

Although the invention has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the invention.

What is claimed is:

1. A micro-sensing element, comprising:
a substrate;
a micro-sensing structure having a plurality of conductive channels disposed on the substrate, wherein each of the conductive channels comprises a sensing part, a conductive wire and an electrode, and the sensing part is electrically connected with the electrode through the conductive wire; and
a covering material covering the substrate and the conductive wires, wherein each of the sensing parts and each of the electrodes are exposed out of the covering material.

2. The micro-sensing element according to claim 1, wherein each of the sensing parts, the conductive wires and the electrodes is made of metal or an alloy.

3. The micro-sensing element according to claim 2, wherein the metal or the alloy comprises gold, nickel, chromium, iridium, palladium, niobium, titanium or platinum.

4. The micro-sensing element according to claim 1, wherein each of the sensing parts, each of the electrodes and each of the conductive wires are integrally formed.

5. The micro-sensing element according to claim 1, wherein the covering material is a polymeric material.

6. The micro-sensing element according to claim 1, wherein an insulating layer is disposed between the conductive channels and the substrate.

7. The micro-sensing element according to claim 1, wherein the substrate is a silicon substrate.

8. A manufacturing method of a micro-sensing element, comprising the steps of:
providing a substrate;
forming at least one micro-sensing structure on the substrate, wherein the micro-sensing structure has a plurality of conductive channels, each of the conductive channels comprises a sensing part, a conductive wire and an electrode, and the sensing part is electrically connected with the electrode through the conductive wire;
covering the substrate and the conductive channels by a covering material; and
partially removing the covering material to make each of the sensing parts and each of the electrodes be exposed out of the covering material.

9. The method according to claim 8, wherein each of the sensing parts, each of the electrodes and each of the conductive wires are integrally formed.

10. The method according to claim 8, wherein the micro-sensing element is formed by using a laser to cut the substrate.

11. The method according to claim 8, wherein the step of exposing the sensing parts comprises the sub-steps of:
forming a conductive part adjacent to each of the sensing parts before the covering material is covered; and
removing the conductive part after the covering material is covered to form a space serving as an opening to expose each of the sensing parts.

12. The method according to claim 11, wherein the sub-step of removing the conductive part is performed by way of etching.

13. The method according to claim 12, wherein the sub-step of removing the conductive part is performed by way of wet etching.

14. A bio-sensing system, comprising:
a micro-sensing element having a substrate, a micro-sensing structure and a covering material, wherein the micro-sensing structure has a plurality of conductive channels disposed on the substrate, each of the conductive channels comprises a sensing part, a conductive wire and an electrode, the sensing part is electrically connected with the electrode through the conductive wire, the covering material covers the substrate and the conductive wires, and each of the sensing parts and each of the electrodes are exposed out of the covering material; and
at least one electronic assembly electrically connected with the electrodes.

15. The bio-sensing system according to claim 14, wherein each of the sensing parts, the conductive wires and the electrodes is made of metal or an alloy.

16. The bio-sensing system according to claim 15, wherein the metal or the alloy comprises gold, nickel, chromium, iridium, palladium, niobium, titanium or platinum.

17. The bio-sensing system according to claim 14, wherein each of the sensing parts, each of the electrodes and each of the conductive wires are integrally formed.

18. The bio-sensing system according to claim 14, wherein the covering material is a polymeric material.

19. The bio-sensing system according to claim 14, wherein an insulating layer is disposed between the conductive channels and the substrate.

20. The bio-sensing system according to claim 14, wherein the electronic assembly is disposed on a circuit board.
